(19) United States
(12) Patent Application Publication
(10) Pub. No.: US 2012/0253190 A1
(43) Pub. Date: Oct. 4, 2012

(54) CONTRAST-ENHANCED ULTRASOUND ASSESSMENT OF LIVER BLOOD FLOW FOR MONITORING LIVER THERAPY

(75) Inventors: Thomas Patrice Jean Arsene Gauthier, Seattle, WA (US); Edward Lam Shang Leen, Beaconsfield (GB)

(73) Assignee: KONINKLIJKE PHILIPS ELECTRONICS N.V., EINDHOVEN (NL)

(21) Appl. No.: 13/499,383

(22) PCT Filed: Sep. 27, 2010
(86) PCT No.: PCT/US10/50326

§ 371 Date: Jun. 12, 2012

(30) Foreign Application Priority Data
Oct. 1, 2009 (US) 61247655

(51) Int. Cl. A61B 8/13

(52) U.S. Cl. 600/431

(57) ABSTRACT

A method for assessing a liver includes acquiring image information including contrast-enhanced ultrasound images of the liver. A location of the main hepatic artery (MHA) and a location of the main portal vein (MPV) of the liver are identified in at least one of the contrast-enhanced ultrasound images of the liver. Time-intensity information corresponding to perfusion of a contrast agent in the MHA and the MPV is obtained. A biomarker index value (BW) which is a function of the time-intensity information corresponding to the perfusion of contrast agent in the MHA and the time-intensity information corresponding to the perfusion of contrast agent in the MPV is determined.

This application claims the benefit of U.S. provisional application Ser. No. 61/247,655, filed Oct. 1, 2009, which is incorporated herein by reference.

The present system relates to a medical imaging system and, more particularly, to a system for assessing liver blood flow to monitor liver therapy.

At present, the monitoring of liver therapy with contrast-enhanced ultrasound (CEUS) has been conducted by identifying a single target hepatic lesion (tumor) and quantifying its blood flow and fractional blood volume. Such a protocol assumes that the response (or absence thereof) to treatment of a single target lesion is representative of the systemic response to treatment, which may not be a valid assumption in all cases.

Accurate early assessment of treatment response is key for efficient management of cancer patients and evaluation of new therapeutic compounds. Histological determination of the mean intra tumor micro vascular density (MVD) is the most commonly used method for assessing angiogenesis. However, MVD measurement not only requires an invasive procedure to obtain tissue, but MVD also does not provide an accurate assessment of the functionality of tumor vessels because poorly functioning or collapsed vessels have endothelial cells that are stained and counted in the analysis. Therefore, determination of changes in MVD may not accurately reflect the effectiveness of anti-angiogenic therapy. For many years, the standard way to assess tumor response to treatment has been to measure tumor size by axial computed tomography (CT) or magnetic resonance imaging (MRI), following either the World Health Organization (WHO) or Response Evaluation Criteria in Solid Tumor (RECIST) guidelines. However, it may take several weeks to months for tumor morphology to noticeably change. These anatomy-based imaging techniques may at best be a lagging indicator of the effectiveness of the treatment regimen. Newly introduced biological anti-cancer compounds, such as anti-angiogenic agents, can cause tumor enlargement before shrinkage, or may stabilize tumor growth to allow patients to live in symbiosis with their cancer. Size criteria are not effective in such cases.

Most cancer treatments currently available do not work immediately, do not cause complete necrosis, and may not dramatically affect tissue characteristics (compared with ablation therapy). It is therefore not straightforward to detect tumor change by current imaging techniques unless the tumor size changes. Quantification of function of the tumor either by blood flow or by metabolism is therefore an attractive method to assess the response to therapy.

Contrast-enhanced ultrasound (CEUS) is one of three leading functional imaging techniques (together with FDG-PET and DCE-MRI) which have been used to assess tumor response to anti-angiogenic therapy. As is well known, PET is the abbreviation for positron emission tomography, and Fludeoxyglucose (¹⁸F) or fluorodeoxyglucose (¹⁸F) is commonly abbreviated as FDG. Further, DCE-MRI is the abbreviation for dynamic contrast enhanced-magnetic resonance imaging.

In previous studies, the quantification of tumor blood flow and tumor fractional blood volume has been performed off-line using two-dimensional (2D) CEUS cine loops. In this scenario, the operator scans in real-time only one plane in the tumor, and then positions a region of interest (ROI) in an image of the tumor. The operator then derives parameters within the identified ROI (e.g. peak intensity, wash-in slope, area under the curve, rise time, mean transit time) from a time-intensity curve which represents contrast uptake by the tumor.

The current monitoring of liver therapy with CEUS performs this same procedure in serial studies at successive points in time during therapy. Using this protocol, slight changes in the scan plane and/or adjustments to the region of interest position from one study to the next may significantly impact time-intensity curve analysis and thereby inaccurately derive parameters which are thought to correlate with tumor blood flow and tumor fractional blood volume. It is also assumed in this scenario that quantitative information derived from the single acquired scan plane is representative of the overall tumor vascularity, which would only be accurate if the tumor was truly homogeneous. Most are not. Also, such a protocol assumes that the response (or absence thereof) to treatment of a single tumor is representative of the systemic response of the liver to treatment, which is also likely to be inaccurate.

When the above protocol is used in the context of tumor therapy monitoring, it is extremely challenging to compare parameter values obtained at different time points during treatment, since it is most likely that the operator is unable to perfectly reproduce the exact same scan plane and region of interest positions. Other challenges associated with using the above protocol for monitoring liver-targeted therapy include finding the same target hepatic lesion in all follow-up scans, where it may have significantly shrunk or become occult to CEUS. Accordingly, there is a need for systems and methods that better assess a tumor's response to therapy and treatment.

One object of the present invention is to overcome the disadvantages of conventional systems, methods, and devices.

In one illustrative embodiment, the present system provides an imaging biomarker which may be used to assess tumor response to treatment. This biomarker may be used to provide an early assessment of whether a particular therapeutic regimen is appropriate or effective following initiation of therapy, and may have implications for both patient management and for drug development to support “go/no-go” (e.g., continue/discontinue treatment or drug development) decisions and accelerate clinical trials.

The present system uses a real-time low mechanical index contrast-enhanced ultrasound (CEUS) imaging mode. Rather than focusing on a single target lesion, the present system images and quantifies the main hepatic arterial and portal venous blood flows to produce an index of their comparative flow characteristics (i.e., a biomarker index value) and thereby assess the hepatic systemic response to treatment and/or condition of the liver.

In accordance with an aspect of the present system, there is disclosed a method for assessing a liver. The method includes an act of acquiring image information comprising a sequence of contrast-enhanced ultrasound images of the liver. The method may further include an act of identifying a location of a main hepatic artery (MHA) and a location of a main portal vein (MPV) of the liver in at least one of the contrast-enhanced ultrasound images of the liver. The method may also include an act of obtaining time-intensity information corresponding to a perfusion of a contrast agent in the MHA and the MPV. Moreover, the method may include an act of determining a biomarker index value (BIV) which is a function of the time-intensity information corresponding to the perfusion of contrast agent in the MHA and the time-intensity information corresponding to the perfusion of contrast agent in the MPV. Based on the BIV, a visual and/or an audible indicator(s) of the condition of the liver is provided to an output device, such as a display or speaker.

According to the method, the BIV may be based upon a ratio of the time-intensity information corresponding to the perfusion of the contrast agent in the MHA to the time-intensity information corresponding to the perfusion of the contrast agent in the MPV. For example, the BIV may be defined as shown in equation (1) below. Thus, the BIV may be defined as:

$$\frac{peak intensity \times wash \text{-} in slope ( main hepatic artery )}{peak intensity \times wash \text{-} in slope ( main portal vein )} ,$$

where:

$$wash \text{-} in slope = \frac{peak intensity}{rise time}$$

of a corresponding MHA and MPV.

This equation corresponds with equation (1) as set forth below.

Further, according to the method, the act of identifying the location of the MHA and the location of the MPV may include an act of receiving location information corresponding to the location of the MHA or the MPV from a user via a user interface (UI) which may include, for example, a keyboard, a touch screen, a voice input, etc., with which a user may enter information. Further, in accordance with the method, the act of identifying the location of the MHA and the location of the MPV may be automatically performed by an image processing algorithm executed by a processor, for example.

Moreover, in accordance with the method, the time-intensity information may be based upon pixel intensity information indicative of each of the perfusion of the contrast agent in the MHA and the perfusion of the contrast agent in the MPV. Further, the method may include an act of automatically introducing the contrast agent into a blood stream of a patient under the control of the processor for obtaining desired images, e.g., using feedback from automatically analyzing the obtained imaged by the processor e.g., using image detection and processing algorithm.

In accordance with another aspect of the present system, there is disclosed a system for assessing a liver, the system includes a processor which acquires image information comprising a sequence of contrast-enhanced ultrasound images of the liver, identifies a location of a main hepatic artery (MHA) and a location of a main portal vein (MPV) of the liver in at least one of the contrast-enhanced ultrasound images of the liver, obtains time-intensity information corresponding to perfusion of a contrast agent in the MHA and the MPV, and/or determines a biomarker index value (BIV) which is a function of the time-intensity information corresponding to the perfusion of contrast agent in the MHA and the time-intensity information corresponding to the perfusion of contrast agent in the MPV. It is also envisioned that the processor may determine the BIV based upon a ratio of the time-intensity information corresponding to the perfusion of the contrast agent in the MHA to the time-intensity information corresponding to the perfusion of the contrast agent in the MPV. Further, the processor may calculate the BIV in accordance with equation (1) below.

Further, it is envisioned that the system may include a user interface (UI) which receives location information corresponding to the location of at least one of the MHA and the MPV from a user and forwards this information to the processor which identifies the location of at least one of the MHA and the MPV based upon the location information. It is also envisioned that the image processing portion may identify the location of at least one of the MHA and the MPV in at least one of the contrast-enhanced ultrasound images of the liver using an image processing algorithm.

According to an embodiment of the present system, the processor may determine the time-intensity information based upon pixel intensity information indicative of the perfusion of the contrast agent in at least one of the MHA and the MPV. Moreover, the system may include an injection portion which may automatically introduce the contrast agent into a blood stream of a patient under the control of the processor, such as using the detected pixel intensity in the processed images as feedback to control the introduction of the contrast agent.

In accordance with yet a further aspect of the present system, there is disclosed a computer program including non-transitory computer instructions stored on a tangible computer readable memory medium and operative to cause the processor to perform various acts. For example, the computer program may be configured to assess liver treatment, the computer program including a program portion configured to acquire image information comprising a sequence of contrast-enhanced ultrasound images of the liver, identify a location of a main hepatic artery (MHA) and a location of a main portal vein (MPV) of the liver in at least one of the contrast-enhanced ultrasound images of the liver, obtain time-intensity information corresponding to a perfusion of a contrast agent in the MHA and the MPV; and/or determine a biomarker index value (BIV) which is a function of the time-intensity information corresponding to the perfusion of contrast agent in the MHA and the time-intensity information corresponding to the perfusion of contrast agent in the MPV.

In accordance with the computer program, the program portion may be configured to determine the BIV based upon a ratio of the time-intensity information corresponding to the perfusion of the contrast agent in the MHA to the time-intensity information corresponding to the perfusion of the contrast agent in the MPV. Further, the program portion may be configured to determine the BIV by calculating in accordance with equation (1) below. Further, the program portion may be configured to identify the location of the MHA and the location of the MPV using location information corresponding to the location of the MHA or the MPV received from a user via a user interface (UI). It is further envisioned that the program portion may be configured to identify the location of the MHA and the location of the MPV using an image processing algorithm. Further, the program portion may be configured to determine the time-intensity information based upon pixel intensity information indicative of each of the perfusion of the contrast agent in the MHA and the perfusion of the contrast agent in the MPV. It is also envisioned that the program portion may be configured to control an injection portion to introduce the contrast agent into a blood stream of a patient.

The invention is explained in further detail, and by way of example, with reference to the accompanying drawings wherein:

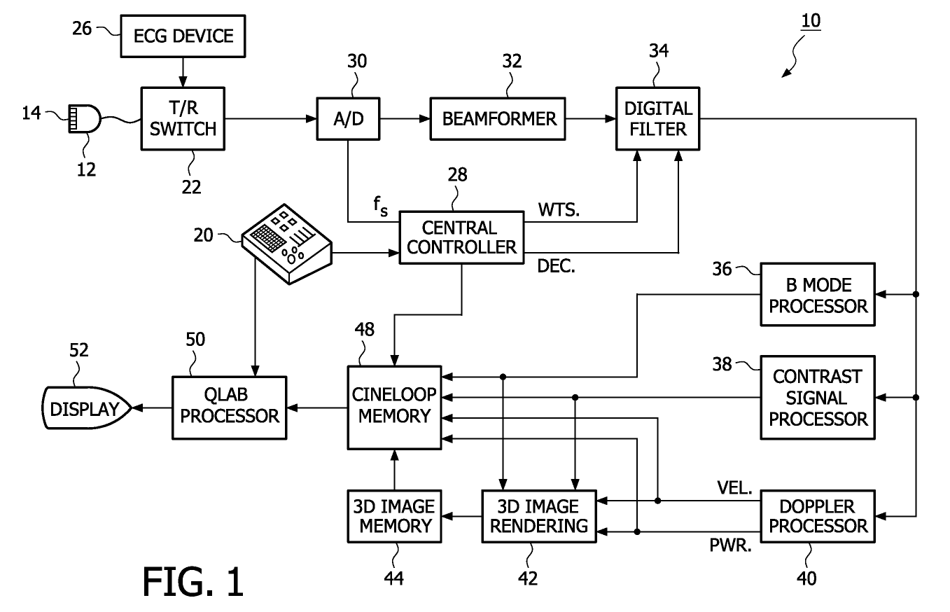

FIG. 1 is a temporal contrast enhanced ultrasound image of portion of a liver in accordance with an embodiment of the present invention.

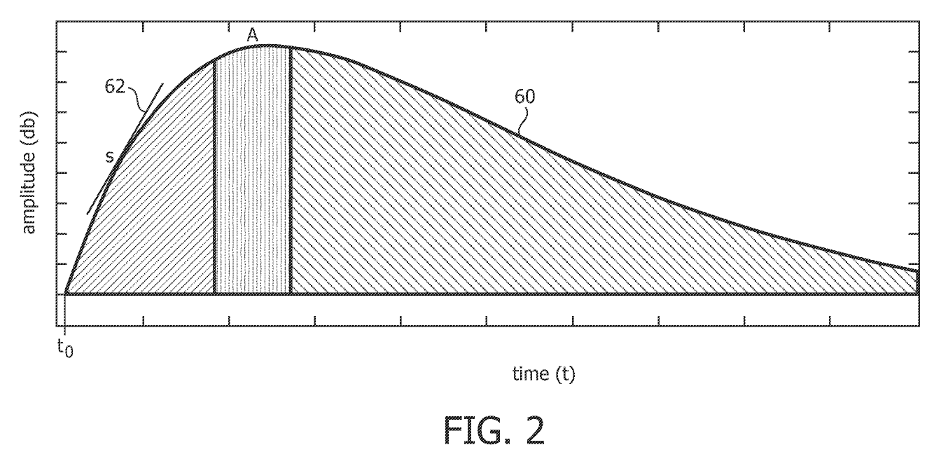

FIG. 2 is a temporal contrast enhanced ultrasound image of a portion of the liver in accordance with an embodiment of the present invention taken a few seconds after the image of FIG. 1.

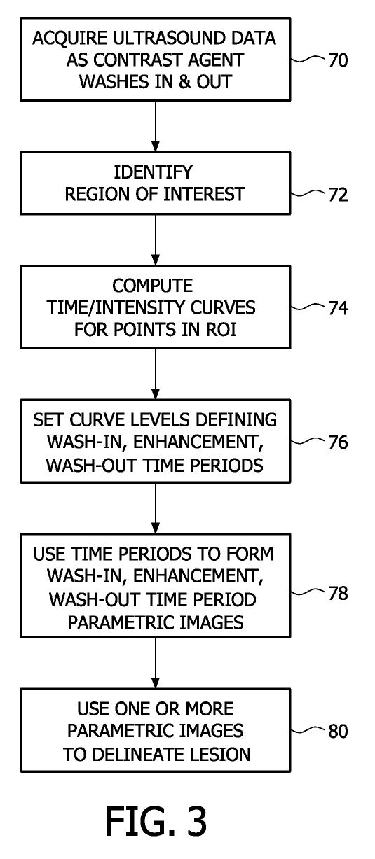

FIG. 3 is a graph showing first and second time-intensity curves generated in accordance with an embodiment of the present invention.

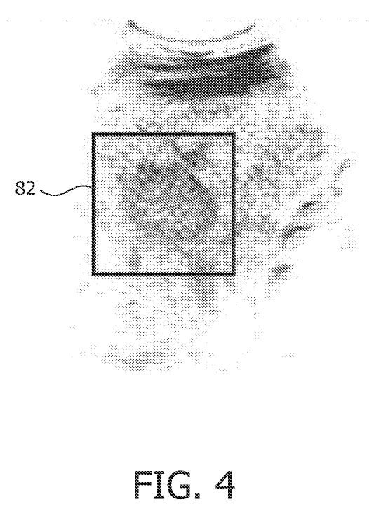

FIG. 4 is a block diagram of a contrast-enhanced ultrasound system in accordance with an embodiment of the present invention.

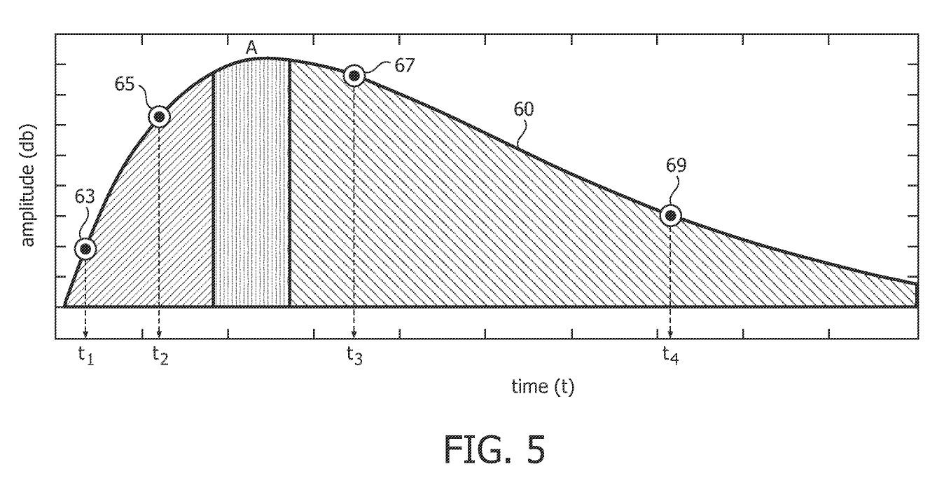

FIG. 5 shows a flow diagram that illustrates a process in accordance with an embodiment of the present invention.

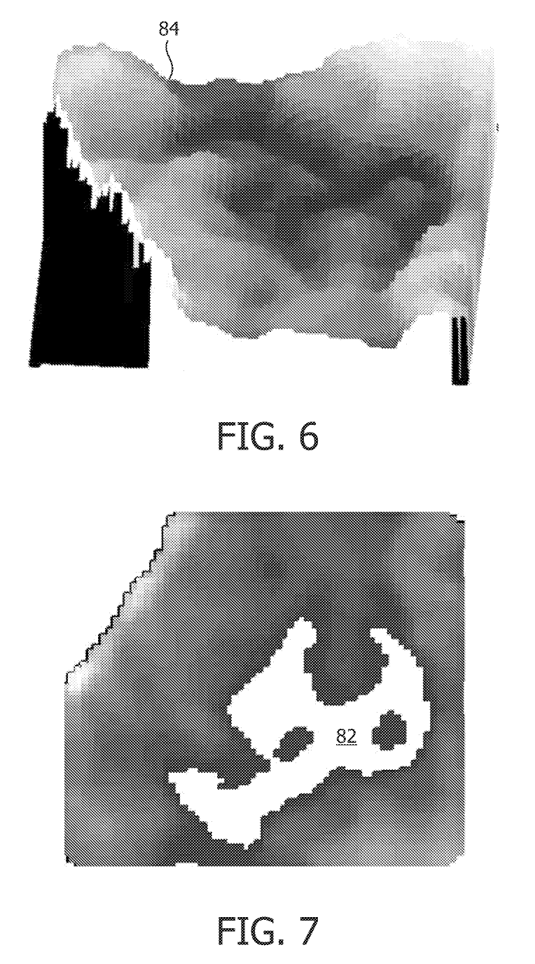

FIG. 6 shows a portion of a system in accordance with an embodiment of the present invention.

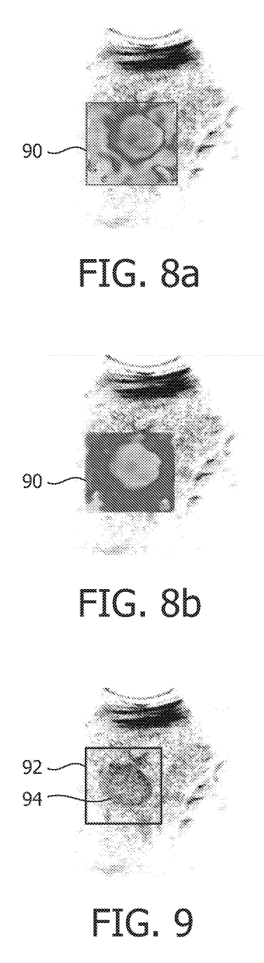

FIG. 7 illustrates a graph of experimental results of a liver assessment study performed by the present invention.

The following are descriptions of illustrative embodiments that when taken in conjunction with the following drawings will demonstrate the above noted features and advantages, as well as further ones. In the following description, for purposes of explanation rather than limitation, illustrative details are set forth such as architecture, interfaces, techniques, element attributes, etc. However, it will be apparent to those of ordinary skill in the art that other embodiments that depart from these details would still be understood to be within the scope of the appended claims. Moreover, for the purpose of clarity, detailed descriptions of well known devices, circuits, tools, techniques and methods are omitted so as not to obscure the description of the present invention. It should be expressly understood that the drawings are included for illustrative purposes and do not represent the scope of the present invention. In the accompanying drawings, like reference numbers in different drawings may designate similar elements.

The present invention may be implemented in an imaging and/or an assessment system which has a real-time low mechanical index CEUS imaging mode to obtain contrast-enhanced images. As the illustrated system incorporates a low mechanical index, disruption to contrast agent microbubbles may be minimized or entirely prevented so that the microbubbles and their passage through blood vessels (such as the blood vessels of a patient) can be accurately imaged, viewed, and/or quantified.

Rather than focusing on a single hepatic target lesion, an embodiment of the present invention may image and quantify main hepatic arterial (hereinafter hepatic artery) and main portal venous (hereinafter portal venous) blood flows to the liver to assess a systemic response of the liver to treatment. US 2009/0124907A1 (Bruce et al.) published on May 14, 2009, and entitled “Ultrasonic Diagnostic Imaging System and Method for Detecting Lesion of the Liver,” the contents of which are incorporated herein by reference, describes how contrast flow in the hepatic artery and portal vein can be used to detect a tumor based, for example, on the time of arrival of the contrast agent the at hepatic artery and the portal vein. The blood supply to a tumor comes primarily from the hepatic artery, which supplies blood with each heartbeat at an earlier time than the flow in the portal vein.

A process to automatically find a hepatic artery and a portal vein will now be discussed with reference to FIG. 1 which is a screen shot that illustrates a temporal contrast enhanced ultrasound image 100 of portion of a liver in accordance with an embodiment of the present invention, and FIG. 2 which is a screen shot that illustrates a temporal contrast enhanced ultrasound image 200 of portion of a liver in accordance with an embodiment of the present invention taken a few seconds after the image 100 of FIG. 1.

In both FIG. 1 and FIG. 2, the left sides of the screen shots show what is referred to as the contrast side, and the right sides of the screen shots show what is referred to as the tissue side. Both the left side and right side images are acquired in parallel, and contrast agents are present when both the left and right sides images are acquired. The difference between the left/contrast side and the right/tissue side is that the signal processing applied by the present system (to form the left/contrast side images) isolates signals from microbubbles of the contrast agent using CEUS imaging, whereas the signal processing applied by the present system to the right/tissue side isolates signals from the tissue alone (and gets rid of signals from the microbubbles). The right/tissue side is similar to a typical ultrasound grayscale B-mode image except that the image may be formed at a much lower mechanical index (which may translate to a much lower acoustic power) than a conventional grayscale B-mode image.

The left/contrast sides of FIGS. 1-2 show blood vessels, and provides dynamic images as the microbubbles of the contrast agent flow through the blood vessels, where the blood with the contrast microbubbles first flows through the main hepatic artery and later through the main portal vein. The image at the left or contrast side in FIG. 1 is used to identify the location of the main hepatic artery 102. As the blood with the contrast microbubbles first flows through the main hepatic artery and then flows through the main portal vein, the images in FIG. 2 are taken later in time after the images shown in FIG. 1, where the location of a main portal vein 104 (where the blood with the contrast agent flows after the flow through the main hepatic artery) is identified in the left contrast side of FIG. 2.

The tissue side is a static image of the same scan plane, since the blood flow is not discernable (where signals from microbubbles are filtered out), and the ultrasound images mainly shows soft tissue. The location of the main hepatic artery 102 and the location of a main portal vein 103, as determined from the dynamic left/contrast side images, may be drawn by the present system on the static right/tissue side images, as shown by the dotted boxes in the right tissue sides of FIGS. 1 and 2. Thus, the images at the right/tissue sides of FIGS. 1-2, that include the drawn locations of the main hepatic artery 102 and the location of a main portal vein 103 may be used to ensure that the scan plane stays in the same location throughout the scan. The advantage of the right side image is that it displays soft tissues which are not changing in appearance over time and thus makes it easier to ensure that the scan plane being imaged by the ultrasound probe remains the same and stays in the same location throughout the scan, whereas the left side image is presenting the dynamic wash-in and wash-out of contrast microbubbles.

Comparing the left contrast side image with the right tissue side image allows a determination of the location of the main hepatic artery 102 and the location of the main portal vein 103 in the same scan plane, where any change in the scan plane is more easily discernable from the static images in right/tissue sides of FIGS. 1 and 2, that include the drawn locations of the main hepatic artery 102 and the main portal vein 103. Using both images from the contrast and tissue sides allows for better positioning and maintaining of an ultrasound probe at the same desired location and provides a sequence of images over time in the same scan plane.

According to an embodiment of the present invention, a process may automatically find the main hepatic artery and/or the main portal vein in a 2D CEUS cine loop. The process may use an image processing algorithm to determine an arrival of contrast agent in the hepatic artery. Thus, the process may use an algorithm which may 1) identify a frame (e.g., an image frame of an image sequence) in a 2D CEUS cine loop where contrast agent first arrives by identifying when, in the 2D cine loop, the intensity of a group of pixels begins to increase from a baseline level and 2) within that particular frame, the algorithm may automatically draw a first region of interest (ROI) around that group of pixels and adjust the position of the first ROI on subsequent frames using a motion estimation technique (e.g., such as the one currently implemented in the QLAB™ ultrasound image analysis package available from Philips™ Healthcare of Andover, Mass.). Motion estimation techniques to select, draw, position, and/or adjust an ROI may used and are known in the art (such as, for example, U.S. Patent Publication No. 2005/0096543 A1 to Jackson et al., filed on Jun. 4, 2004 and entitled “Motion Tracking For Medical Imaging,” the contents of which is incorporated herein by reference) and will not be discussed further for the sake of clarity. The first ROI may then be used to identify the first group of pixels in other frames in the image sequence. Thus, pixels within the first ROI will correspond with the first group of pixels. Further, the first ROI may correspond with the hepatic artery.

To determine whether the intensity of the first group of pixels has begun to increase from the baseline level, the process may compare the intensity of the first group of pixels with a baseline threshold value (e.g., a predetermined value or an average value of an intensity of the first group of pixels over a certain time period such as a time period before contrast agent is injected), and when it is determined that the intensity of the first group of pixels is equal to or greater than the threshold value, the process may determine that the first group of pixels has begun to increase from the baseline level. Conversely, when the intensity of the first group of pixels is determined to be less than the baseline threshold value, the process may determine that the intensity of the first group of pixels has not begun to increase from the baseline level.

Referring to FIG. 1, a hepatic artery 102 is shown in the image 100 and a corresponding ROI_{HA} 103 has been automatically selected by the system. The portal vein is always in the vicinity of the hepatic artery and pixels corresponding with a location of the portal vein will light up with the arrival of contrast agent a few seconds (e.g., in subsequence frames of the image sequence) after the hepatic artery does. Accordingly, the image processing algorithm may take advantage of knowing anatomically where to look for the portal vein (given that the hepatic artery has already been identified at this point and that it is known that the portal vein is in the vicinity of the hepatic artery), and draw a corresponding ROI (i.e., a second ROI) around a group of pixels (hereinafter a second group of pixels) which corresponds with the location of the portal vein. The process may select the second group pixels corresponding with the location of the portal vein by, for example, determining when the intensity of the second group of pixels begins to increase from a baseline level. As this process may be similar to the process described above with respect to the selection of the first group of pixels (e.g., corresponding with the hepatic artery), for the sake of clarity a further description thereof will not be provided. The process may identify the second group pixels in the image sequence using, for example, the second ROI as the second group of pixels will be in the second ROI in other frames in the image sequence, e.g., of the 2D CEUS cine loop.

After drawing the second ROI, the algorithm may adjust the position of the second ROI on subsequent frames using the motion estimation technique discussed above with respect to the first ROI.

It is also envisioned that the image processing algorithm may also take advantage of the B-mode 2D cine loop (e.g., available in a contrast side-by-side image presentation) where the portal vein is usually very easily identifiable given its anechoic lumen and bright interfaces and define a corresponding ROI_{PV}.

Referring to FIG. 2, a portal vein 104 is shown in the image 200 and a corresponding second ROI 105 which has been automatically selected by the system is seen in close proximity to the first ROI 103.

It is further envisioned that the first and second ROIs and/or other ROIs may be defined by a user and/or manually drawn. For example, the user may manually draw ROIs on regions corresponding with the hepatic artery, the portal vein, blood vessels, etc. using predefined shapes such as circles, rectangles, etc., which may be provided on a display of the system and may be selected and thereafter placed over a desired location in an image frame to define a corresponding ROI. Thus, for example, a user may manually draw a ROI on, for example, a blood vessel by selecting a shape and placing the selected shape over the vessel. The system may then store the corresponding ROI with image information for later analysis.

Moreover, according to yet other embodiments, a real-time three-dimensional (3D) CEUS image sequence may be used to ensure acquisition of a large enough volume in the liver to contain the main hepatic artery and portal vein, and the system may then scan and select a particular plane where both the hepatic artery and portal vein are visible. The process may then use image information corresponding with the selected plane to perform one or more processes which assess a systemic response of the liver to treatment in accordance with an embodiment of the present invention.

Accordingly, the present invention may provide an operator-independent system for the detection of main liver vessels and/or subsequent quantitative analysis of the liver as will be described below.

After the groups of pixels (e.g., the first and second groups of pixels corresponding with the first and second ROIs, respectively) are selected by the system or the user as describe above, the system may determine a corresponding intensity of pixels in the first and second sets of pixels, respectively, over time and form corresponding contrast intensity information (e.g., first and second contrast intensity information, respectively).

FIG. 3 is a graph 300 showing first and second time-intensity curves 302 and 304, respectively, generated in accordance with an embodiment of the present invention. First contrast intensity information 303 (e.g., comprising connected points) corresponds with the first contrast intensity information over time, and second contrast intensity information 305 corresponds with the second time-intensity information over time. The first and second contrast information values may be processed, e.g., by averaging, fitting, etc., using any suitable algorithm by the system to respectively form the first and second time-intensity curves 302 and 304, respectively. Accordingly, the first time-intensity curve 302 corresponds with (e.g., by fitting, averaging, etc.) the first contrast intensity information 303 and is, thus, related to the flow of contrast agent in the hepatic artery over time. Similarly, the second time-intensity curve 304 corresponds with (e.g., by fitting, averaging, etc.) the second contrast intensity information 305 and is, thus, related to the flow of contrast agent in the portal vein over time.

Accordingly, the first time-intensity curve 302 may correspond with the intensity of the first group of pixels (e.g., in the first ROI) and may be related to the flow of contrast agent in the hepatic artery over time. Similarly, the second time-intensity curve 304 may correspond with the intensity of the second group of pixels (e.g., in the second ROI) and may be related to the flow of contrast agent in the portal vein over time.

In accordance with an embodiment of the present invention, a biomarker index value (BIV) may be used to determine a hepatic systemic response to treatment of the liver. Accordingly, the biomarker index value may be determined based upon a flow of contrast agent in the hepatic artery and portal vein over time. The process may determine that a hepatic systemic response to treatment (e.g., of the liver) is deemed to be effective when the process determines that the biomarker index value is less than or equal to a biomarker threshold value (BTV). However, if the process determines that the biomarker index value is greater than the threshold value BTV, the process may determine that the hepatic systemic response to treatment is not deemed to be effective.

Accordingly, the process may alert a user of the findings or only parts thereof, such as by displaying a message on a display indicating the response to treatment is, or is not, effective depending on the value of the biomarker index in comparison with the BTV. For example, the process may alert a user using a first alert method to call the user's attention to the exam results (e.g., by highlighting exam results or other audio visual method) when it is determined that the hepatic systemic response to treatment is not deemed to be effective. However, when it is determined that the hepatic systemic response to treatment is deemed to be effective, the process may display this information with the exam results.

In accordance with an embodiment of the present invention, the biomarker index value (e.g., an imaging biomarker) comprises a contrast-enhanced perfusion index (CEPI) value which is a function of the flow characteristics of contrast agent in both the hepatic artery and the portal vein over time. Accordingly, the CEPI value may be derived from the first and second time-intensity curves 302 and 304, respectively, and may be a ratio defined as shown in Equation (1) below. In accordance with the present embodiment, the biomarker index value is equal to the CEPI value. However, alternatively or in addition to using a ratio of certain parameters, such as the CEPI, it is also envisioned that the biomarker index value includes other parameters, such as differences or products of flow parameters. A CEPI equation of the present invention is:

$$CEPI = \frac{peak {intensity}_{1} \times wash \text{-} in {slope}_{1}}{peak {intensity}_{2} \times wash \text{-} in {slope}_{2}} \\ where , \\ wash \text{-} in {slope}_{1} = \frac{peak {intensity}_{1}}{rise {time}_{1}} , \\ and \\ wash \text{-} in {slope}_{2} = \frac{peak {intensity}_{2}}{rise {time}_{2}} \qquad Eq . ( 1 )$$

In Equation (1), the peak intensity₁ corresponds with a first peak intensity 306 of the first time-intensity curve 302, shown in FIG. 3, (e.g., corresponding with the flow of contrast agent in the hepatic artery) and may be automatically determined by a process of the present system. Similarly, the peak intensity₂ corresponds with a second peak intensity 308 of the second time-intensity curve 304 (e.g., corresponding with the flow of contrast agent in the portal vein) and may be automatically determined by a process of the present system. With reference to rise-times, the rise-time₁ corresponds with a first rise time 310 for the first time-intensity curve 302 (e.g., a rise time of the hepatic artery) and may be defined as (t2−t1), where t1 is a time at which the first time-intensity curve 302 is determined to exceed the baseline level and t2 is a time at which the first peak intensity 306 occurs. Similarly, the rise-timeₜ corresponds with a second rise time 312 for the second time-intensity curve 304 (e.g., a rise time of the portal vein) and may be defined as (t4−t3), where t3 is a time at which the second time-intensity curve 304 is determined to exceed the baseline level and t4 is a time at which the second peak intensity 308 occurs.

Using a ratio eliminates various effects, such as using different amounts of contrast agent, which affects various parameters such as the amplitude of the peaks of both intensity curves 302, 304 for the main hepatic artery and the main portal vein, where using a ratio eliminates such effects and compensates the use of different amounts of contrast agent from one exam to the next, for example.

The first contrast intensity information 303 is related to the flow of contrast agent in the hepatic artery over time and may be acquired by the system as a bolus of contrast agent arrives at the hepatic artery and begins to flow through and build up in the vessels of the liver. Similarly, the second contrast intensity information 305 is related to the flow of contrast agent in the portal vein over time and may be acquired by the system as a bolus of contrast agent arrives at the portal vein and begins to flow through and build up in the vessels of the liver. The time-intensity curves (e.g., 302 and 304) may then be produced from the contrast intensity information as is known in the art. See, for example, WO 2010/055426A1 (Chang) published on May 20, 2010 and entitled “Ultrasonic Lesion Identification Using Temporal Parametric Contrast Image;” and WO 2009/093211A1 (Averkiou et al.) published on Jul. 30, 2009, and entitled “Therapy Assessment with Ultrasonic Contrast Agents,” where the contents of each are incorporated herein by reference.

Generally, the peak intensity value of a corresponding time-intensity curve is multiplied by the wash-in slope of that curve, where the wash-in slope is determined from the peak intensity and rise time of the curve as shown in FIG. 3.

It is further envisioned that, instead of the BIV being a ratio as defined by the CEPI, the BIV may comprise other combinations of the flow characteristics of the hepatic artery and portal vein which may alternatively be used to produce a biomarker value, such as different ratios, or differences or products of flow parameters. For example, it is envisioned that a BIV maybe calculated as a ratio of the wash-in slope of the hepatic artery divided by wash-in slope of the portal vein (wash-in slope₁/wash-in slope₂); or may be calculated based on t4−t2, i.e. the difference in time between the two peaks 308, 306 shown in FIG. 3. For example, the value t4−t2 is compared to a difference threshold value (which may be experimentally derived) for assessing the condition of the liver and/or whether or not the treatment is effective (such as when value of the difference t4−t2 is greater or less than the difference threshold).

Conventional systems image and analyze a single tumor which provides inaccurate results as typically many tumors may be present in the liver. Instead of imaging and analyzing a single tumor, the present system analyzes the whole liver using blood flow though the main hepatic artery and the main portal vein using serial measurements or a sequence of images taken over a period of time.

Typically, a healthy liver receives the majority of the blood, e.g., approximately 75% of the blood, from the main portal vein and approximately 25% from the main hepatic artery. More and more blood is delivered through the main hepatic artery for a diseased or unhealthy liver, where approximately 75% of the blood may be from the main hepatic artery and approximately 25% from the main portal vein, for example. Accordingly, the source of the blood for the liver provides an indication of the health of the liver and a threshold value or BTV is determined depending on the particular type of BIV or marker used. If the marker or BIV is defined by equation (1), than the BTV is determined to be 12.57 based on experimental or statistical data and/or studies (e.g., the study associated with FIG. 7) to discriminate between patients with healthy and unhealthy (e.g., cancerous) livers.

FIG. 7 illustrates a scatter plot of experimental results of a liver assessment study performed by an embodiment of the present invention where the dots represent persons. The study was performed on a plurality of persons and found that a biomarker index value of the present system (such as the value of CEPI) has excellent sensitivity and specificity to the effectiveness of liver tumor therapy performed on a subset of the plurality of persons in the study. In particular, FIG. 7 shows a scatter plot of measurements of the biomarker index value (BIV) as defined in equation (1) in healthy controls (group A—healthy liver), subjects with untreated liver metastases (group B—abnormal liver), and responders, i.e. subjects with liver metastases who were successfully treated (group C—successfully treated liver). The number of subjects or dots in FIG. 7 is 20 healthy controls (group A), 25 subjects with untreated liver metastases (group B), and 7 responders (group C). The threshold value used is 13 and is shown by a horizontal line. Thus, subjects with a BIV less than or equal to BTV are classified as having healthy livers, while subjects with a BIV greater than the BTV are classified as having unhealthy livers.

During the study, a biomarker index value was calculated for the liver of each person (i.e., dots in FIG. 7), in accordance with a method of the present invention, from image sequence loops of the hepatic artery and portal vein. Each of the plurality of persons was also grouped as a member of first through third groups in accordance with a liver assessment. The members of the first group have malignant hepatic lesions (e.g., see ‘abnormal’ group in FIG. 7), the members of the second group have benign hepatic lesions (e.g., see ‘normal’ group in FIG. 7), and the members of the third group have cancer which has gone into remission (e.g., see ‘post treatment’ group in FIG. 7). The biomarker index values of the first group were clearly segmented from those of the second and third groups in the study.

Further, the process may classify a person into a predetermined group by comparing the biomarker index value or BIV of the corresponding person with a threshold value and classifying the person based upon the result of the comparison. Thus, for example, if the biomarker index value of a person undergoing liver therapy is determined to be equal to or less than the threshold value, the assessment of the liver therapy for the corresponding person may be determined to be successful. However, if the biomarker index value of the person is greater than the threshold value, the assessment of the liver therapy for that person or patient may be determined to be unsuccessful and a new therapy may be administered. Then, the patient may be monitored in a similar fashion, where the therapy assessment is repeated to determine whether this new treatment is successful. Results of the process may be stored for later use and/or evaluation.

Using the BTV to classify subjects as having healthy or unhealthy livers based on the respective BIV values provides an excellent diagnostic tool. Further, the trend of the BIV also provides an assessment of the therapy. For example, if the BIV decreases after a particular therapy or treatment of the liver, then the decreasing BIV (even if the BIV is still above the BTV) indicates that this particular treatment is effective and increasing the health of the liver. If the BIV is not changing or increasing, then this indicates that the particular treatment is either ineffective or detrimental to the health of the liver.

FIG. 4 is a block diagram of an ultrasound system 10 for contrast-enhanced liver diagnosis in accordance with an embodiment of the present invention. The ultrasound system 10 may perform one or more techniques in accordance with an embodiment of the present invention. An ultrasonic probe 12 includes an array 14 of ultrasonic transducers that transmits and receives ultrasonic signals. The array 14 may be a one dimensional linear or curved array for two dimensional imaging, or may be a two dimensional (2D) matrix of transducer elements for electronic beam steering in three dimensions. The array 14 may also be a one dimensional array that is mechanically swept back and forth by the ultrasonic probe 12 to scan a three dimensional volume of the body. The ultrasonic transducers in the array 14 transmit ultrasonic energy and receive echoes returned in response to this transmission. A transmit/receive (“T/R”) switch 22 is coupled to the ultrasonic transducers in the array 14 to selectively couple signals from the transducer elements to A/D converters 30 during the receive phase of operation. The times at which the array 14 is activated to transmit signals may be synchronized to an internal system clock, or may be synchronized to a bodily function such as the heart cycle, for which a heart cycle waveform is provided by an electrocardiography (ECG) device 26. When the heartbeat is at the desired phase of its cycle as determined by the waveform provided by the ECG device 26, the ultrasonic probe 12 is commanded to acquire an ultrasonic image. In the conduct of the present invention, a continuous sequence of real time images may be acquired as blood containing contrast agent begins to flow through the hepatic artery and the portal vein.

Echoes from the transmitted ultrasonic energy are received by the transducers of the array 14, which generate echo signals that are coupled through the T/R switch 22 and digitized by analog to digital (“A/D”) converters 30 when the system uses a digital beamformer. Analog beamformers may alternatively be used. The A/D converters 30 sample the received echo signals at a sampling frequency controlled by a signal fs generated by a central controller 28. The desired sampling rate dictated by sampling theory is at least twice the highest frequency of the received passband, and might be on the order of 30-40 MHz. Sampling rates higher than the minimum requirement are also desirable. Control of the ultrasound system and the setting of various parameters for imaging, such as probe selection, are affected by user manipulation of the controls of the user interface of a control panel 20 which is coupled to and applies its control through the central controller 28.

The echo signal samples from the individual transducers of the array 14 are delayed and summed by a beamformer 32 to form digital coherent echo signals. For 3D imaging with a two dimensional array, it is preferable to partition the beamformer 32 between a microbeamformer located in the ultrasonic probe 12 and the main beamformer in the system mainframe as described in U.S. Pat. No. 6,013,032 (Savord) and U.S. Pat. No. 6,375,617 (Fraser), the contents of each of which are incorporated herein by reference. The digital coherent echo signals are then filtered by a digital filter 34. In this embodiment, the transmit frequency and the receiver frequency are individually controlled so that the beamformer 32 is free to receive a band of frequencies which is different from that of the transmitted band such as a harmonic frequency band for detection of harmonic contrast agents. The digital filter 34 bandpass filters the signals, and can also shift the frequency band to a lower or baseband frequency range. The digital filter 34 could be a filter of the type disclosed in U.S. Pat. No. 5,833,613 (Averkiou et al.), for example, the contents of which are incorporated herein by reference. Filtered echo signals from tissue are coupled from the digital filter 34 to a B mode processor 36 for conventional B mode processing and the production of 2D B mode images.

Filtered echo signals of a contrast agent, such as microbubbles, are coupled to a contrast signal processor 38. Contrast agents are often used to more clearly delineate blood vessels, or to perform perfusion studies of the microvasculature of tissue as described in U.S. Pat. No. 6,692,438 (Skyba et al.) for example, the contents of which are incorporated herein by reference. The contrast signal processor 38 preferably separates echoes returned from harmonic contrast agents by the pulse inversion technique, in which echoes resulting from the transmission of multiple pulses to an image location are combined to cancel fundamental signal components and enhance harmonic components. A preferred pulse inversion technique is described in U.S. Pat. No. 6,186,950 (Averkiou et al.), for instance, the contents of which are incorporated herein by reference.

The filtered echo signals from the digital filter 34 are also coupled to a Doppler processor 40 for conventional Doppler processing to produce velocity and/or power Doppler images. The output signals from these three processors 36, 38, 40 may be scan converted and displayed as planar 2D images, and are also coupled to a 3D image processor 42 for the rendering of three dimensional (3D) images, which are stored in a 3D image memory 44. Three dimensional rendering may be performed as described in U.S. Pat. No. 5,720,291 (Schwartz), and in U.S. Pat. Nos. 5,474,073 (Schwartz et al.) and 5,485,842 (Quistgaard), the contents of each of which are incorporated herein by reference.

The two dimensional (2D) image signals from the contrast signal processor 38, the B mode processor 36 and the Doppler processor 40, and the three dimensional (3D) image signals from the 3D image memory 44 are coupled to a memory such as a Cineloop™ memory 48, which stores image data for each of a large number of ultrasonic images. The image data are preferably stored in the Cineloop™ memory 48 in sets, with each set of image data corresponding to an image obtained at a respective time. The image data in a sequence of images can be used to display a parametric image showing tissue perfusion at a respective time during the heartbeat. In this embodiment, the images are also coupled to a QLAB™ processor 50, where the images are analyzed and quantified measurements made of flow characteristics of the hepatic artery and portal vein as described above, to generate a biomarker such as the biomarker index value described above. A time-intensity curve for the hepatic artery is produced from contrast pixel data of a sequence of images of the hepatic artery as the contrast agent arrives at, builds up and declines in the hepatic artery. A similar time-intensity curve is produced for the portal vein. The image sequences used to produce the two curves may be slightly different as the contrast flow in the hepatic artery may be better imaged in a given image sequence and the contrast flow in the portal vein may be better imaged in another image sequence. Both image sequences are sub-sequences of the same continuous image sequence. Image processing techniques such as respiratory gating to overcome the effects of motion (see, e.g., international patent application no. PCT/IB09/050,277 filed Jan. 23, 2009 and entitled “Respiratory-Gated Therapy Assessment with Ultrasonic Contrast Agents”, the contents of which are incorporated herein by reference) may be applied to isolate hepatic artery-specific image sub-sequences and portal vein-specific image sub-sequences. The contrast-enhanced perfusion index (CEPI) described above is then calculated from the data of the two time-intensity curves using equation (1).

The QLAB™ processor is a software package that is commercially available with Philips™ Healthcare ultrasound systems for various image analysis and quantification procedures. The QLAB™ processor can be used to make quantified measurements of various aspects of the anatomy in the image such as the delineation of tissue boundaries and borders by automated border tracing as described in US patent publication no. US2005-0075567 and PCT publication no. WO2005/054898, the contents of each of which are incorporated herein by reference. The QLAB™ processor is controlled through user manipulation of controls such as buttons and a trackball of the control panel 20. The data and images produced by the QLAB™ processor, such as those of FIGS. 1-3 above, may be output to an output or rendering device such as a display 52 where the user may locate ROIs on the hepatic artery and portal vein either manually or automatically by image segmentation, and manipulate, annotate and make measurements of biomarkers such as the biomarker index value (BIV or CEPI) of the displayed images through operation of the controls of the control panel 20 as described above.

An assessment portion 54 may receive the biomarker and may compare it with a threshold value to assess the liver and/or effectiveness of the treatment in accordance with the various embodiments as described, such as the in accordance with the operation described in connection with FIG. 5. Results of the assessment may be used by the QLAB™ processor to be rendered on the display 52, processed, and/or stored on a memory of the ultrasonic system 10.

FIG. 5 shows a flow diagram that illustrates a process 500 in accordance with an embodiment of the present system, where operational acts of the process 500 may be performed by the processor 50 executing non-transitory computer instructions stored on a tangible computer readable memory medium, such as a memory coupled to or accessible by the processor 50, including the memories 44, 48 of the ultrasound system 10, or other memories such as hard drives and/or removable memories or storage medium, such as optical discs, and/or remote memories coupled to servers accessible through a network such as the Internet. The process 500 may be performed using one or more computers communicating over a network. The process 500 can include one of more of the following acts. Further, one or more of these acts may be combined and/or separated into sub-acts, if desired. In operation, the process may start during act 501 and then proceed to act 503.

During act 503, the process may obtain a temporal image sequence of a liver captured using a contrast-enhanced ultrasonic imaging system. The image sequence may be captured in real-time or may be obtained from a memory of the system. The image sequence should span a sufficient period of time to obtain desired frames. After completing act 503, the process may continue to act 505.

During act 505, the process may determine first and second regions of interest (ROI-1 and ROI-2, respectively). The first region of interest in the image sequence may correspond with a first group of pixels associated with a location of the hepatic artery, and the second region of interest may correspond with a second group of pixels associated with a location of the portal vein in the image sequence. After completing act 505, the process may continue to act 507.

During act 507, the process may determine first and second intensity information for the first and second regions of interest, respectively. Accordingly, the process may determine first and second intensity information for the first and second groups of pixels, respectively. After completing act 507, the process may continue to act 509.

During act 509, the process may process may generate first and second time-intensity curves (e.g., see intensity curves 302 and 304, respectively) based upon the first and second intensity information, respectively. Accordingly, the process may process the first and second intensity information using any suitable algorithm (e.g., moving average, curve fitting, etc.) and may generate corresponding first and second time-intensity curves, respectively. After completing act 509, the process may continue to act 511.

During act 511, the process may process information related to the first time-intensity curve (e.g. 302) and determine a peak intensity (e.g., a maximum value) and a corresponding rise-time (e.g., for the hepatic artery). The rise-time may correspond with an interval such as the interval (t241) or rise time 310 as shown in FIG. 3, where t2 corresponds with a peak time at the peak (e.g., maximum) intensity of the first time-intensity curve, and t1 corresponds with a baseline time at which it is determined that the intensity of the first group of pixels (e.g., corresponding with the first region of interest) is equal to or greater than a first threshold value. After completing act 511, the process may continue to act 513.

During act 513, the process may process information related to the second time-intensity curve (e.g., 304) and determine a peak intensity (e.g., a maximum value) and a corresponding rise-time (e.g., for the portal vein). The rise-time may correspond with an interval such as the interval (t4−t3) as shown in FIG. 3, where t4 corresponds with a peak time at the peak intensity of the second time-intensity curve, and t3 corresponds with a second baseline time at which it is determined that the intensity of the second group of pixels (e.g., corresponding with the second region of interest) is equal to or greater than a second threshold value. After completing act 513, the process may continue to act 515. With respect to the first and second threshold values, these values may be the same as, or different from, each other.

During act 515, the process may determine the biomarker index value, such as a contrast-enhanced perfusion index (CEPI) value, in accordance with equation (1) above. After completing act 515, the process may continue to act 517. During act 517, the process may compare the biomarker index value (BIV) with a biomarker index threshold value (BTV). Accordingly, if the process determines that the biomarker index value or BIV is less than or equal to the biomarker index threshold value or BTV, the process may continue to act 519. However, if the process determines that the biomarker index value or BIV is greater than the biomarker index threshold value or BTV, the process may continue to act 525.

The BTV may include one or more values such as a value of 12.57 which is selected in an embodiment of the present invention where the marker is defined by equation (1) to maximize and enhance sensitivity and specificity. However, it is envisioned that the BTV may include other values which may be may be set by the system or the user based upon various information, e.g., type of marker, user's age, sex, disease type, treatment type, and the like. As described, different markers may be used, such as a marker associated with or indicating t4−t2, which is the difference in time between the two peaks 308, 306 of the two curves shown in FIG. 3, namely the intensity curve 304 corresponding with the flow of contrast agent in the portal vein and the intensity curve 302 corresponding with the flow of contrast agent in the hepatic artery. If the marker is associated with or indicates t4−t2, for example, then another threshold value (other than 12.57) may be determined and used where, based on the determined threshold value, a patient is classified as a responder or as a non-responder to the therapy. Threshold values for different markers may be refined and/or determined experimentally based on collected data for specific sub-populations based on, e.g., sex, age, disease type, treatment type, etc. During act 519, the process may set a current liver assessment marker (e.g., a flag, a bit, a word, etc.) as positive. This may indicate that the liver under assessment is, for example, responding to a current treatment regimen or therapy or meets a certain criteria, such as CEPI being less than or equal to BTV, or other threshold value. After completing act 519, the process may continue to act 521.

During act 525, the process may set a current liver assessment marker as negative. This may indicate that the liver under assessment is, for example, not responding to a current treatment regimen or therapy or does not meet a certain criteria, such as CEPI being greater than BTV, or other threshold value. After completing act 525, the process may continue to act 521.

During act 521, the process may render results of the assessment. For example, the process may indicate that the current liver assessment is positive or negative based on, for example, the results of the determination of act 517 by providing a visual and/or audio indication to an output or rending device, such as displaying (on the display 52) a green indicator or message that the treatment is effective, or a red indicator or message that the treatment is not effective. An audio alert by also be provided through a speaker to alert personnel, such as physicians, nurses or technicians that the treatment is not effective, for example. After completing act 521, the process may continue to act 523.

During act 523, the process may update and/or store information corresponding with the current assessment. Accordingly, the process may store information generated by the current process such as, for example, the assessment findings, ROIs, contrast intensity information, peak intensity values, rise times (e.g., hepatic artery and portal vein), biomarker index value, current assessment marker, etc., for later use. After completing act 523, the process may continue to act 527, where it ends.

FIG. 6 shows a portion of a system 600 (e.g., including server(s) connected to network(s) with nodes connected to each other directly or through networks, using wired or wireless connections) in accordance with an embodiment of the present system. For example, a portion of the present system may include a processor 610 operationally coupled to a memory 620, a display 630, a transducer 614, an ECG 626, an injector 656 and a user input device 670. The memory 620 may be any type of device for storing application data as well as other data related to the described operation. The application data and other data are received by the processor 610 for configuring (e.g., programming) the processor 610 to perform operation acts in accordance with the present system. The processor 610 so configured becomes a special purpose machine particularly suited for performing in accordance with the present system.

The operation acts may include requesting, providing, and/or rendering of content. The user input 670 may include a keyboard, mouse, trackball or other device, including touch sensitive displays, which may be stand alone or be a part of a system, such as part of a personal computer or other device for communicating with the processor 610 via any operable link. The user input device 670 may be operable for interacting with the processor 610 including enabling interaction within a UI as described herein. Clearly the processor 610, the memory 620, display 630, the ECG 626, the transducer 614, the injector 656, and/or user input device 670 may all or partly be a portion of a computer system or other device such as a client and/or server as described herein.

The processor 610 is operable for providing control signals and/or performing operations in response to input signals from the user input device 670 as well as in response to other devices of a network and executing instructions stored in the memory 620. The processor 610 may be an application-specific or general-use integrated circuit(s). Further, the processor 610 may be a dedicated processor for performing in accordance with the present invention or may be a general-purpose processor wherein only one of many functions operates for performing in accordance with the present invention. The processor 610 may operate utilizing a program portion, multiple program segments, or may be a hardware device utilizing a dedicated or multi-purpose integrated circuit.

The transducer 614 may include an array of ultrasound transducers and may obtain contrast enhanced image information under the control of the processor 610. The ECG 626 may generate a synchronization signal in accordance with a bodily function (e.g., a heart cycle, etc.) of a patient and may output this signal to the processor 610. Accordingly, when the processor 610 determines that the heartbeat of the patient is at the desired phase of its cycle as determined by the waveform provided by an ECG device 266, the processor 610 may command the ultrasonic probe 12 (FIG. 4) to acquire an ultrasonic image of the patient (e.g., the patient's liver).

The injector 656 may include an apparatus to inject contrast agent into the patient's body in order to introduce the contrast agent into the patient's bloodstream in order to perform a CEUS imaging technique in accordance with the present invention. Accordingly, the injector 656 may include a reservoir to hold a desired amount of contrast agent and a motor portion to pump the contrast agent into the blood stream of the patient under the control of the processor 610.

Further variations of the present system would readily occur to a person of ordinary skill in the art and are encompassed by the following claims. Through operation of the present system, a virtual environment solicitation is provided to a user to enable simple immersion into a virtual environment and its objects.

What is claimed is:

1. A method for assessing a condition of a liver, the method comprising:
acquiring image information comprising contrast-enhanced ultrasound images of the liver using an ultrasonic probe;
identifying a location of a main hepatic artery (MHA) and a location of a main portal vein (MPV) of the liver in at least one of the contrast-enhanced ultrasound images of the liver;
obtaining time-intensity information corresponding to a perfusion of a contrast agent in the MHA;
obtaining time-intensity information corresponding to a perfusion of a contrast agent in the MPV;
determining a biomarker index value (BIV) as a function of the time-intensity information corresponding to the perfusion of contrast agent in the MHA and the time-intensity information corresponding to the perfusion of contrast agent in the MPV; and
outputting to an output device an indication of the condition of the liver based on the BIV.

2. The method of claim 1, wherein the BIV is based upon a ratio of the time-intensity information corresponding to the perfusion of the contrast agent in the MHA to the time-intensity information corresponding to the perfusion of the contrast agent in the MPV.

3. The method of claim 1, wherein the BIV is defined as:
$$BIV = \frac{peak intensity \times wash \text{-} in slope ( main hepatic artery )}{peak intensity \times wash \text{-} in slope ( main portal vein )}$$
where:
$$wash \text{-} in slope = \frac{peak itensity}{rise time}$$
of a corresponding MHA and MPV.

4. The method of claim 1, wherein the act of identifying the location of the MHA and the location of the MPV comprises receiving location information corresponding to the location of the MHA or the MPV from a user via a user interface.

5. The method of claim 1, wherein the act of identifying the location of the MHA and the location of the MPV is automatically performed by a processor performing image processing of the contrast-enhanced ultrasound images.

6. The method of claim 1, wherein the time-intensity information is based upon pixel intensity information indicative of each of the perfusion of the contrast agent in the MHA and the perfusion of the contrast agent in the MPV.

7. The method of claim 1, further comprising an act of introducing the contrast agent into a blood stream of a patient.

8. A system for assessing a condition of a liver, the system comprising:
a processor which is configured to:
acquire image information comprising contrast-enhanced ultrasound images of the liver;
identify a location of a main hepatic artery (MHA) and a location of a main portal vein (MPV) of the liver in at least one of the contrast-enhanced ultrasound images of the liver;
obtain time-intensity information corresponding to a perfusion of a contrast agent in the MHA;
obtain time-intensity information corresponding to a perfusion of a contrast agent in the MPV; and
determine a biomarker index value (BIV) as a function of the time-intensity information corresponding to the perfusion of contrast agent in the MHA and the time-intensity information corresponding to the perfusion of contrast agent in the MPV.

9. The system of claim 8, wherein the processor is further configured to determine the BIV based upon a ratio of the time-intensity information corresponding to the perfusion of the contrast agent in the MHA to the time-intensity information corresponding to the perfusion of the contrast agent in the MPV.

10. The system of claim 1, wherein the processor determines the BIV using a following equation:
$$BIV = \frac{peak intensity \times wash \text{-} in slope ( main hepatic artery )}{peak intensity \times wash \text{-} in slope ( main portal vein )}$$
where:
$$wash \text{-} in slope = \frac{peak itensity}{rise time}$$
of a corresponding MHA and MPV.

11. The system of claim 8, further comprising a user interface which receives location information corresponding to the location of at least one of the MHA and the MPV from a user and forwards the location information to the processor for identifying the location of at least one of the MHA and the MPV based upon the location information.

12. The system of claim 8, wherein the processor is further configured to perform image processing for identifying the location of at least one of the MHA and the MPV in at least one of the contrast-enhanced ultrasound images of the liver.

13. The system of claim 8, wherein the processor is further configured to determine the time-intensity information based upon pixel intensity information indicative of the perfusion of the contrast agent in at least one of the MHA and the MPV.

14. The system of claim 8, further comprising an injection device, wherein the processor is further configured to control the injection device to automatically introduces the contrast agent into a blood stream of a patient.
